(19) United States
(12) Patent Application Publication
(10) Pub. No.: US 2026/0283506 A1
(43) Pub. Date: Sep. 24, 2026

(54) CALCULATION MODULE CONFIGURED TO EVALUATE VISUAL AND POSTURAL ERGONOMICS

(71) Applicant: Essilor International, Charenton-Le-Pont (FR)

(72) Inventors: Konogan BARANTON, Charenton Le Pont (FR); Aurélie LE CAIN, Charenton Le Pont (FR); Delphine TRANVOUEZ-BERNARDIN, Charenton Le Pont (FR); Jean SAHLER, Charenton Le Pont (FR)

(73) Assignee: Essilor International, Charenton-Le-Pont (FR)

(21) Appl. No.: 19/480,216

(22) PCT Filed: Apr. 11, 2024
(86) PCT No.: PCT/EP2024/059818

§ 371 Date: Oct. 30, 2025

(30) Foreign Application Priority Data
May 4, 2023 (EP) 23315162.0

(51) Int. Cl. A61B 5/11; A61B 5/00

(52) U.S. Cl. CPC A61B 5/1116; A61B 5/1114; A61B 5/1121; A61B 5/486; A61B 5/6803; A61B 5/7264

(57) ABSTRACT

A calculation module for evaluating visual and postural ergonomics of a user and issuing recommendations, the calculation module being configured to: obtain at least one value of at least one optical parameter representative of at least one optical element worn by the user, obtain at least one value of at least one positional parameter representative of a position, a movement or a succession of movements of at least one body-part of the user, select reference values for the at least one positional parameter based on the value of the at least one optical parameter, compare the values of the at least one positional parameter to the selected reference values to detect an inadequacy, and issue at least one recommendation to the user in case of a detected inadequacy.

### FIELD OF THE INVENTION

The invention relates to the field of visual and postural ergonomics.

More specifically, the invention relates to a calculation module configured to evaluate visual and postural ergonomics of a user and issue recommendations.

The invention also relates to a computer-implemented method implemented on such a calculation module.

### BACKGROUND OF THE INVENTION

The concern for improved postural and visual ergonomics is tied to the rise of the sedentary lifestyle and the consequences of poor posture associated with it. Both in a work environment and in a personal environment, people spend large amounts of time looking at of screens of computers, televisions or smartphones, and often in less than adequate postures. Notably, head position, neck angles, shoulder posture, etc, may lead to imbalances and irritation in muscles and tendons. This may lead to chronic discomfort or pains that can be avoided with a reinforced attention to posture and by devoting sufficient time to postural adjustments or break from screen-related activities.

Furthermore, visual ergonomics also encompass addressing visual discomfort, which may be a consequence of a prolonged attention to a bright screen with insufficient breaks, or an excess of near-field vision with insufficient periods of far-field accommodation to compensate. Lack of attention to visual ergonomics may lead to deterioration in visual disorders such as myopia, as well as general discomfort, such as migraines.

Moreover, these negative effects are increased in case of a bad adequation between the recommended use of ophthalmic devices, notably progressive ophthalmic lenses, and their usage by the wearer. Indeed, progressive lenses have separate viewing areas dedicated to near-field and far-field vision, and an improper use by looking through the wrong area may lead to an increased visual discomfort as described above.

Visual and postural training with a professional and/or physician often leads to improvements in comfort and solves many of these issues. However, the adjustments to visual and postural behavior must become habits to ensure prolonged beneficial effect, and thus require frequent reinforcement to provide their maximum benefit. As the attention of the person is often devolved to the activity being practiced, the care to posture and vision ergonomics declines and harmful habits often overtake the benefits of previous training.

### SUMMARY OF THE INVENTION

The invention aims at solving the aforementioned problems by providing an everyday, easily implemented way to obtain feedback and recommendation relative to postural and visual ergonomics.

To this end, the invention relates to a calculation module for evaluating visual and postural ergonomics of a user and issuing recommendations, the calculation module being configured to:
obtain at least one value of at least one optical parameter representative of at least one optical element worn by the user,
obtain at least one value of at least one positional parameter representative of a position, a movement or a succession of movements of at least one body-part of the user,
select reference values for the at least one positional parameter based on the value of the at least one optical parameter,
compare the values of the at least one positional parameter to the selected reference values to detect an inadequacy, and
issue at least one recommendation to the user in case of a detected inadequacy.

Such a device provides continued feedback and recommendations towards visual and postural ergonomics of the user, allowing a continuous and easily implemented reinforcement of good practice to improve postural and visual comfort.

The at least one optical element may comprise, for example, an optical lens, notably an ophthalmic lens.

The calculation module may be placed on a head-mounted device.

The head-mounted device may also comprise the at least one optical element.

The head-mounted device may be, for example, a set of smart glasses.

Alternatively, the control module may be separate from the head-mounted device and connected to the head-mounted device through a wired or wireless connection.

The values of the at least one optical parameter may be obtained from a memory.

The memory may be placed on the head-mounted device or a separate device, such as a computer or smartphone. The memory may be connected to the calculation module directly or through a wireless communication protocol.

The values of the positional parameter(s) may be obtained through measurements by at least one sensor.

The at least one sensor may comprise an image sensor, such as a camera, an inertial sensor, a gyroscopic sensor, a localization sensor such as a GPS, etc.

Different sensors from among the at least one sensor may be used to provide values of different environmental and/or postural parameters.

The at least one sensor may be placed on the head-mounted device.

According to an implementation, the at least one positional parameter comprises at least one angle, direction or distance of observation of an area of interest by the user.

The positional parameter may be obtained, for example, by acquiring at least one image with an eye-tracking camera.

This feature allows the device to provide feedback relative to the use of any eventual ophthalmic corrective equipment worn by the user, as well as on maintaining a correct visual posture relative to their activity.

According to another implementation, the reference values comprise a range of adequate distances, angles, or directions of observation of an area of interest seen by the user through the optical element.

This feature allows to check correct use of any ophthalmic correcting lens worn by the user. For example, the calculation module is configured to check that the user is watching an item in a near-field of vision using the correct ophthalmic corrective lens.

According to another implementation, the optical element is a progressive ophthalmic lens, the range of the adequate directions or the angles of observation of the area of interest being determined in relation to a distance to the area of interest and in relation to specific predetermined areas of the optical element.

This feature allows a control over the adequation between the viewing distance to an area of interest and the used area of the progressive lens that provides the adequate visual correction for this viewing distance.

According to another implementation, the area of interest comprises a display, the calculation module being configured to select the reference values also using a distance to the display. This feature allows a control over viewing distance to a display and allows the calculation module to issue recommendation to the user to adjust this distance if inadequate.

The display may be a static display, such as a text page or book, or a dynamic display, such as a screen.

According to another implementation, the display comprises at least one text area, the calculation module being configured to select the reference values also using a parameter representative of the text area.

The parameter representative of the text area may be, for example, a font size, a luminosity, and/or a contrast value.

This feature allows the identification of a text on a display and the recommendation of adjustment to the display in case of inadequate text parameters, or the adjustment of the distance to the display or to the environment conditions such as ambient luminosity.

According to another implementation, the at least one positional parameter comprises at least one head or neck position angle, the at least one reference parameter comprising a corresponding recommended angular range, the at least one reference parameter further comprising a maximal duration spend out of the recommended angular range over a predetermined duration.

This feature allows a control over improper body posture, notably head and neck posture, for example when watching a screen.

According to another implementation, the at least one positional parameter comprises a frequency of postural alteration, the postural alteration comprising a variation of at least one predetermined body-part positional angle by a predetermined minimum value, the at least one reference parameter comprising a minimum frequency of postural alteration, and/or wherein the at least one positional parameter comprises a frequency of movement alteration, the movement alteration comprising a variation of at least one predetermined body-part movement by a predetermined minimum value, the at least one reference parameter comprising a minimum frequency of movement alteration.

This feature allows to check a sufficient frequency of breaks in the current activity and postural changes, such a getting up after sitting in front of a screen for a certain duration.

According to another implementation, the recommendation comprises a recommended displacement of the area of interest and/or a recommended displacement of the user relative to the area of interest.

This feature allows to check for adequate viewing position of an object and correction of this relative position if needed.

According to another implementation, the recommendation comprises a recommended postural alteration.

The recommended postural alteration may comprise instructions to move at least one specific body part of the user to improve a posture of the user.

According to another implementation, the recommendation comprises a postural alteration, the postural alteration comprising a variation of at least one predetermined body-part positional angle by a predetermined minimum value, a recommended break from a current activity, and/or a modification of the optical element.

This feature allows to recommend changes in body posture on case of a prolonged inadequate posture, or a change of the optical element for a new optical element more adapted to the viewing distance.

According to another implementation, the calculation module is configured to control at least one signaling device for issuing the recommendation, the signaling device comprising a visual device, an audio device, and/or a haptic device.

This feature allows a direct issuance of the recommendation to the user through a simple signal.

The signaling device may be placed on the head-mounted device.

The signaling device may be connected to the control module through a direct wire connection or through a wireless communication protocol.

According to another implementation, the head-mounted device comprises a communication device for communicating with a computer, a smartwatch or a smartphone, for storing measurement data on the computer, the smartwatch or the smartphone and issuing notification through a program running on the computer, the smartwatch or the smartphone.

This feature allows to provide complete feedback over a predetermined length of time of postural and visual behavior of the user, to make long-term recommendations.

According to another implementation, the calculation module is also configured to use at least one environmental parameter comprising a nature of an activity performed by the user, the calculation module being configured to determine the activity being performed by the user based on measurements from at least one sensor, advantageously through an artificial intelligence-type algorithm implemented on the calculation module.

This feature allows an improvement of the accuracy of the selection of the reference values by the device based on the determined nature of the current activity.

According to another implementation, the head-mounted device is configured to generate reference values of the at least one postural parameter associated and corresponding values of the at least one environmental parameter in a calibration step, the calibration step being triggered by the user or automatically.

This feature allows a generation and correction of the database based on the user's individual behavior and characteristics and thus improves the adequation of the reference values used. The invention also relates to a head mounted device comprising a calculation module and at least one optical element,
the calculation module being configured to evaluate visual and postural ergonomics of a user wearing the optical element and issue recommendations,
the calculation module being configured to:
obtain, at least one value of at least one optical parameter representative of the optical element,
obtain, at least one value of at least one positional parameter representative of a position, a movement or a succession of movements of at least one body-part of the user,
select reference values for the at least one positional parameter based on the value of the at least one optical parameter,
compare the values of the at least one positional parameter to the selected reference values to detect an inadequacy, and
issue at least one recommendation to the user in case of a detected inadequacy.

The invention also relates to a computer-implemented method for evaluating visual and postural ergonomics of a user and issuing recommendations, the method comprising steps of:
obtaining at least one value of at least one optical parameter representative of an optical element used by the user,
obtaining at least one value of at least one positional parameter representative of a position, a movement or a succession of movements of at least one body-part of the user,
selecting reference values for the at least one positional parameter based on the value of the at least one optical parameter,
comparing the values of the at least one positional parameter to the selected reference values to detect an inadequacy, and
issuing at least one recommendation to the user in case of a detected inadequacy.

### BRIEF DESCRIPTION OF THE DRAWINGS

FIG. 1 is a perspective view of a head-mounted device comprising a calculation module after the invention,

FIG. 2 is a side view of a user wearing the head-mounted device of FIG. 1,

FIG. 3 is a view of specific areas of a progressive lens, and

FIG. 4 is a schematic representation of a computer implemented method according to the invention.

### DETAILED DESCRIPTION OF EMBODIMENTS

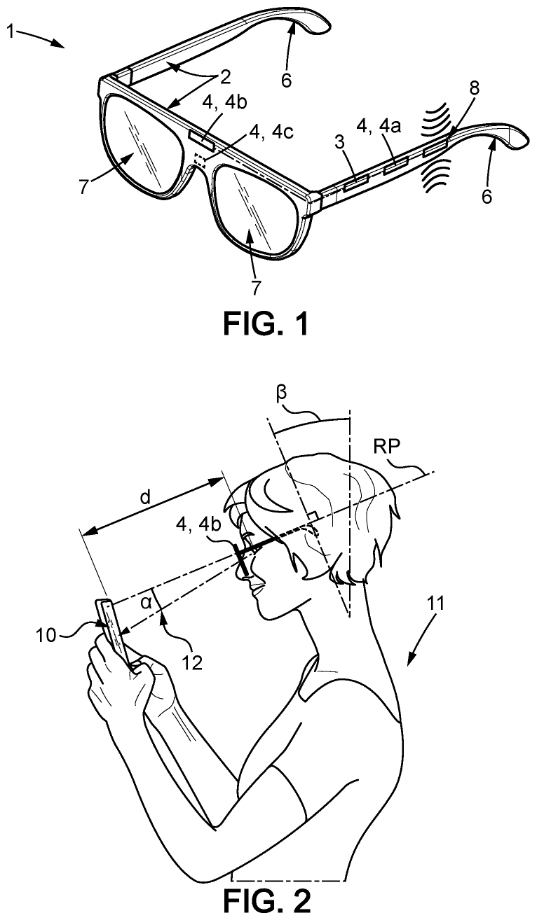

A first implementation of the invention is described below, in reference to FIG. 1. In this implementation, a calculation module 3 according to the invention is mounted on a head-mounted device 1 of the type commonly designated as smart glasses, shown on FIG. 1.

Such a head-mounted device 1 typically comprises a rigid frame 2 comprising a single part or several articulated parts, arranged for being put on the face of a wearer by resting on at least one facial feature such as the nose and/or ears.

The head-mounted device 1 comprises the electronic calculation module 3 and at least one sensor 4 for obtaining information representative of a position and/or of movements of the user of the head-mounted device 1.

The calculation module 3 comprises an electronic circuit comprising at least one processor, for executing programs, and a memory for storing data and instructions for the execution of said programs.

Each sensor 4 is able to measure information relative to the environment of the user, and or to a movement or position of the user. The calculation module 3 is connected to each sensor 4 on the device 1 for piloting the sensor 4 and for analyzing and saving the obtained information.

The at least one sensor 4 may comprise at least one inertial motion sensor 4a (also known as an IMU, for Inertial Motor Unit), which measures linear acceleration (i.e. acceleration in a straight line), a gyroscopic sensor, which measures angular movements and positions, a magnetic sensor, for measuring orientations relative to a reference direction, as well as any sensor that measures information that can be related to movement or position.

The at least one sensor 4 may also include at least one camera mounted on the device 1 and associated with appropriate image treatment algorithm.

Advantageously, the at least one sensor 4 comprise at least one front-facing camera 4b configured to acquire images of the environment in front of the user and at least one eye-tracker camera 4c, configured to acquire images of the eyes of the user to determine a gaze direction through an image treatment method.

The at least one sensor 4 may also comprise a GPS unit, configured to determine a location of the user.

The head-mounted device 1 may also comprise at least one notification device for issuing notifications to the user.

The notification device may for example comprise an audio and/or visual alarm device, a visual display device and/or a haptic device.

The head-mounted device 1 may also comprise a communication device 8, or communication interface, for enabling communication between the head-mounted device and other electronic devices such as a computer, a smartphone, or other elements in a personal area network of the user.

The communication device 8 may function through a direct wire connection or through a wireless connection protocol, such as “Wi-Fi” or “Bluetooth”, for example.

The device typically also comprises a battery 5 for providing electrical power to the calculation module 3 and to each sensor 4.

The calculation module 3 is configured to obtain at least one value of at least one optical parameter representative of at least one optical element 7 worn by the user.

In embodiments, the value of the optical parameter representative of the optical element may evolve. In this case, the calculation module 3 may be configured to obtain the evolved value and to issue the recommendation to the user based on the evolved value.

In the example described, the optical element 7 is an optical lens mounted on the head-mounted device 1.

Preferentially, the optical element 7 is an ophthalmic lens providing an ophthalmic correction to the user. The optical element 7 has at least one optical parameter relative to an ophthalmic correction implemented, values of the optical parameter(s) being saved in the memory of the calculation module 3.

The optical parameter may be an optical power of the ophthalmic lens, or any other parameter representative of an optical correction implemented by the lens, such as a spherical value, a cylinder value, etc . . .

In the case where the optical element 7 is a progressive ophthalmic lens or an anti-fatigue ophthalmic lens (for example the lenses commercialized under the name “Eyezen”, provided by the applicant and presenting an optimisation of the design of the lens on two points, one of the two points being dedicated to a near vision posture and another of the two points being dedicated to a far vision posture), the optical parameter may be a domain of the lens, represented by a range of viewing directions through the lens, associated with a range of viewing distances for which the progressive lens provides an adequate correction when used through the domain.

Alternatively, the optical element 7 may be a plano lens, providing no ophthalmic correction. An ophthalmic lens or a plano lens may also be conformed to modify an intensity of the light traversing the lens, such as a colored absorbing lens, or sunglasses with a gradient of transmission.

Alternatively, the lens may be configured to have variable transition properties, such as a variable transmission lens, presenting light absorption properties that vary either automatically when exposed to incident light, or under control of the calculation module 3.

Alternatively, the optical element 7 may be an electrochromic lens comprising an electrochromic element.

The optical element 7 may have at least one optical parameter relative to a transmission of light through the optical element, such as a transmission coefficient through a fixed or variable transmission lens, or a range of filtered light for a filtering lens, or a polarization angle for a polarizing lens.

Alternatively, the optical element 7 may be separate from the head-mounted device bearing the calculation module.

The optical element 7 may be an ophthalmic lens mounted on a standard pair of glasses, or a contact lens placed directly against an eye of the user.

The optical parameter may be obtained from a database based on measurements by at least one sensor under control of the calculation module.

The calculation module 3 is also configured to obtain at least one value of at least one positional parameter representative of a position, a movement or a succession of movements of at least one body-part of the user.

In other words, the positional parameter is a parameter of a position of a part of the body of the user, for example, a position of the head or the eye of the user. The position may be a statical position or a dynamic position for example a movement a succession of movements of the part of the body of the user. The position of the part of the body may be determined in an absolute manner or in a relative manner, for example, relative to another part of the body or an object held by the user, used by the user or on which the user glazes.

The positional parameter is obtained through at least one measurement by any combination of the at least one sensor 4 controlled by the calculation module 3.

As shown on FIG. 2, the positional parameter may be, for example, descriptive of the observation of an area of interest 10 by the user 11.

The area of interest 10 is for example located in the front field of the user 11, and comprises one or more elements towards which the user's gaze 12 is directed. The elements in the area of interest may be detected in the environment of the user by the sensor 4, for example through acquisition of at least one image by the camera 4b and the use of an image analysis program to detect predetermined specific elements.

The positional parameter may be a distance of observation d separating the area of interest 10 from the user 11, or a direction or an angle a of observation of the area of interest by the user, taken between the gaze direction 12 and at least one reference direction or reference plane RP.

The reference plane may be, for example, a sagittal plane, a transverse plane or a frontal plane relative to the user's head.

The gaze direction 12 is for example determined based on measurements from the eye-tracking camera 4c.

The area of interest 10 may comprise one or more displays, such as for example a screen of a computer or a smartphone, or a static display such as a page comprising written text and/or an image.

The calculation module may be configured to determine a viewing distance separating the user form the display.

The calculation module 3 may be configured to detect the text in the area of interest and obtain at least one text related parameter, such as a font size, a contrast factor, a luminosity, etc . . . This text parameter may be used by the calculation module 3 as a supplemental parameter to the optical parameter(s) and/or positional parameter(s).

The positional parameter may also be representative of a posture of the user 11. In this case, the values of the positional parameter(s) describe the relative positions of at least one body part of the user relative to each other and relative to the area of interest 10.

For example, the positional parameter may be relative to a neck, head and/or shoulder position, such as a position angle of the neck β shown on FIG. 2.

The positional parameter may be calculated from any combination od angles and distances of relative body parts to evaluate a safety of a posture of the user if maintained for a prolonged period, for example.

The at least one positional parameter may also comprise a frequency of postural alteration and/or movement alteration.

The postural alteration for example comprises a variation of at least one predetermined body-part positional angle by a predetermined minimum value.

The evaluation of the frequency of postural alteration may be used to detect a prolonged static posture that would not be unhealthy in itself but should not be maintained for too long to not incur musculo-squeletal discomfort. For example, even a correct and safe sitting position should not be maintained for hours without standing up for the user's comfort.

The movement alteration for example comprises a variation of at least one predetermined body-part movement by a predetermined minimum value.

The frequency of movement alteration is used to detect monotonous and/or repetitive movements that may constitute a musculo-squeletal risk if maintained for too long.

The frequency of postural alteration or of movement alteration is determined by counting how many times the positional angles or positional angles movements vary, by a value higher than the minimum value, in a predetermined time period.

The calculation module 3 is configured to select reference values for the at least one positional parameter based on the value of the at least one optical parameter, in order to compare the values of the at least one positional parameter to the selected reference values to detect an inadequacy.

The reference values are for example taken from a database. The database may be stored in the memory of the calculation module 3 or accessed by the calculation module 3 through the communication interface 8.

The calculation module 3 is configured to select the reference values based on the measured values of the optical parameters. The reference values may also be determined based on values of any complementary parameter, such as the viewing distance and/or the text parameter above.

The reference values may comprise a range of adequate distances, angles, or directions of observation of an area of interest seen by the user through the optical element 7, selected based on the values of the at least one parameter.

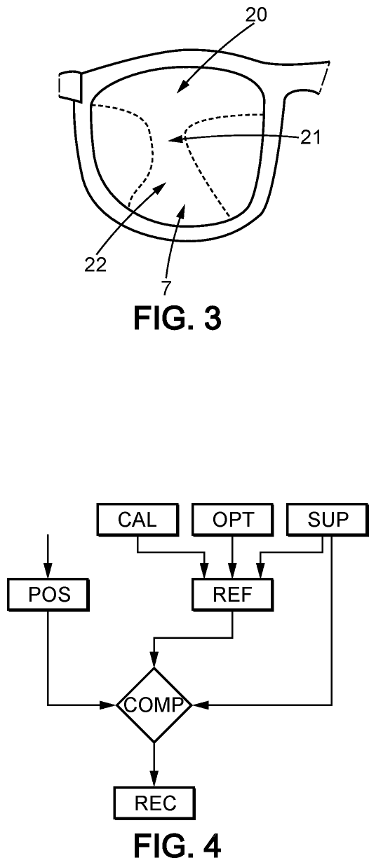

Notably, when the optical element is a progressive ophthalmic lens or bifocal lens, as shown on FIG. 3, the range of the adequate directions or the angles of observation of the area of interest is determined in relation to a distance to the area of interest and in relation to specific predetermined areas of the optical element 7.

As shown on FIG. 3, such an optical element 7 comprises a region adapted for far-field vision 20, generally on an upper part of the lens, a region for intermediate field vision 21 and a region for near-filed vision 22, generally located on a lower part of the lens.

Then, depending on the determined viewing distance, an adequate region of the lens is determined, and compared to the detected gaze direction to check correct use of the progressive lens.

Indeed, incorrect use of a progressive ophthalmic lens by observing elements through a region of the lens that is unsuitable for the viewing distance may cause visual discomfort.

In another implementation, the reference values may include, for example, a recommended angular range for a position angle of the head, neck and/or shoulder and a maximum duration spend outside of this recommended angular range.

The comparison of the measured values and reference values of the positional parameter may comprise the calculation of a risk score by the calculation module.

Such a risk score is for example calculated by a preset algorithm taking into account an angle of head flexion or extension, an angle of head lateral bending, a duration spend in a static posture or a repetitive behavior of the user, such as a frequency of adopting the posture.

The risk score takes for example minimal values, associated with low risk, in case of low head flexion or extension associated with low head lateral flexion and a low frequency of repetition and short durations spend in the posture.

The risk score may take a high value, associated with a high risk, in the case of a severe head flexion (for example over 45 degrees of flexion) or extension (for example over 20 degrees of extension) associated with a severe head lateral bending (for example over 20 degrees) and a prolonged duration (for example over a minute) or high frequency (for example over 50% of a preset duration).

The reference values of the at least one positional parameter may also comprise a minimum frequency of postural alteration, and/or a minimum frequency of movement alteration.

The inadequacy is determined when a sufficient divergence between the reference values and the measured values of the positional parameter is detected by the calculation module.

A margin for triggering the determination of an inadequacy may be used by the calculation module, depending on the type of values considered as the positional parameter. The margin may be taken from a database, for example stored in the memory.

The inadequacy may also be determined when the measured values of the positional parameter show a divergence from the reference values for a predetermined time period. The predetermined time period for triggering the determination of the inadequacy depends on the type of positional parameter used and may be taken from a database.

The reference values of the positional parameter may comprise a type of movement of the user detected through the motion sensors controlled by the calculation module, and be associated with an average viewing distance that is adequate to the type of optical element 7 worn by the user.

The link between the viewing distance and the type of movement may be determined in a statistical manner by computing the probability of vision at various distances against the type of motor activity in a specific detectable environment.

In this case, a variation of the distribution of viewing distance detected by the sensors compared to the distribution considered as the reference value will be assimilated as an inadequacy.

The calculation module 3 is configured to issue at least one recommendation to the user in case of a detected inadequacy.

The recommendation may be delivered instantaneously and/or be a delayed recommendation, possibly included in a periodic feedback given to the user.

The calculation module 3 may be configured to communicate the recommendation to a central electronic device for preparing the periodic feedback, for example a computer or a smartphone. The recommendation may include a signal or alarm for warning the user of the inadequacy and to suggest a correction to implement.

The signal may be a visual signal, for example delivered through a light or a display mounted on the head-mounted device 1.

Alternatively, the visual signal may be delivered by altering temporarily a property of the optical element 7 in a recognizable pattern.

For example, when the optical element 7 comprises an electrochromic glass, the recommendation may include a variation of a coloration of at least a part of the electrochromic glass to signal the user.

The visual signal may also comprise information directed towards correcting the inadequacy. For example, the signal may comprise a text message identifying the inadequacy and suggesting a correction to be implemented.

For example, in the case of improper use of progressive ophthalmic lenses, the appropriate regions of the lens that should be used in the current situation may be designated visually, for example by a change in color or absorption of the appropriate regions.

In another example, the recommendation may comprise a recommended displacement of an area of interest and/or a recommended displacement of the user relative to the area of interest, to adjust a non-optimal viewing distance to an area of interest.

For example, for a myopic user, the recommendation would be used to avoid prolonged use of the near-filed vision to prevent degradation of myopia.

For example, the visual signal may comprise a pictogram displayed on the smart glasses indicating to increase or decrease the viewing distance accordingly.

Alternatively, in the case of an LED delivering a visual signal, a color of the LED may indicate the correction to be implemented. For example, a red light may signal to increase the viewing distance, and a blue light to reduce the viewing distance.

The signal may also include an auditive component, transmitted by aerial conduction or bone conduction, such as a preregistered alarm sound. The alarm sound may be specific of the type of inadequacy and/or contain information directed toward the correction to be implemented. The signal may also comprise a haptic component, such as a vibration of a haptic device in contact with or in close proximity of the user.

In the case of a posture-related inadequacy, the recommendation may comprise a postural alteration recommendation, the postural alteration comprising a variation of at least one predetermined body-part positional angle by a predetermined minimum value.

This recommendation may be issued through a visual or audio signal that persists until adequate postural changes are made, for example.

The recommendation may also include a simple postural alteration, such as for example prompting the user to stand up after detecting a prolonged sitting period.

The simple postural alteration may also comprise a suggestion to straighten a neck position to prevent a prolonged neck misalignment when reading or watching a screen.

The recommendation may also comprise a recommended break from a current activity and/or a modification of the optical element.

For example, a prolonged use of a screen without appropriate protective lenses may cause discomfort. The calculation module 3 may thus be configured to issue a recommendation to stop watching a screen for a minimum break duration.

The recommendation may also comprise a suggestion to decrease overall time spend watching a screen based on a detected time spend watching a screen during several consecutive days and compared to a reference value of advised maximum screen usage.

The maximum advised screen usage may vary depending on age of the user and the use of protective eyewear, for example.

The maximum advised screen usage may also vary base on a preregistered optometric profile of the user. For example, a myopic condition of the user may be the cause of a lower screen usage recommendation, especially in younger users.

The recommendation may also include a prompt to adjust reading conditions in relation with a text parameter. For example, the recommendation may comprise a suggestion to increase viewing distance to the text, increase ambient luminosity, or to increase the font size when applicable.

The recommendation may also comprise a modification of the optical element. For example, the recommendation may comprise a prompt to put on a pair of spectacles, or to change the spectacles currently in use.

Alternatively, the recommendation may comprise a prompt to use protective glasses, such as sunglasses or blue light filtering glasses, for example.

The frequency or duration prior to issuance of a recommendation, or the type of recommendation, may vary depending on the risk associated with the determined inadequacy. For example, in the case of a calculated risk score, good or acceptable posture associated with a low risk score may lead to no recommendation being issued, a moderate risk score may lead to a recommendation being issued in the periodic report with suggestion to address the inadequacy, while a high risk score indicative of a harmful posture may lead to the issuance of an immediate recommendation through an alarm to prevent prolonged exposition of the user. The calculation module may also be configured to use at least one environmental parameter comprising a nature of an activity performed by the user in selecting the reference values of the positional parameter. The calculation module is configured to determine the activity being performed by the user based on measurements from the at least one sensor, advantageously through an artificial intelligence-type algorithm implemented on the calculation module.

For example, an image treatment program may be used on images of the front field of the user to determine the activity being performed.

Positional measurements, for example through a GPS type sensor, may also be used to determine a location of the user and determine an associated activity, such as for example driving a vehicle, use public transportation, or a sport type activity.

In an implementation of the invention, the head-mounted device is configured to generate reference values of the at least one postural parameter associated and corresponding values of the at least one environmental parameter in a preliminary calibration step, the calibration step being triggered by the user or automatically.

For example, the calculation module 3 is configured to collect values of the positional parameters and any supplementary parameter during the calibration period.

The collected values are used to build a database, with eventual input from the user and/or another qualified operator to select values most associated with an adequate use.

The database comprises reference values of the positional parameter directly calculated or extrapolated from the collected values or the positional parameters during the calibration step. In another implementation of the invention, the calculation module 3 is not positioned on a head-mounted device 1. The calculation module 3 may be integrated to another device worn or carried by the user, such as a smartphone, or to a fixed device, such as a computer. The method realized by the calculation module 3 could be also realized in the cloud, or a split/spread between the smart eyeglasses, the smartphone and the cloud backend.

In this case, the calculation module in in communication with the at least one sensor 4 through a wired or wireless communication device 8. The sensors 4 may comprise fixed sensors, such as a webcam oriented towards the user and connected to a computer, for example, or sensors worn by the user, such as a smartwatch, for example.

The invention also relates to a computer-implemented method for evaluating visual and postural ergonomics of the user and issuing recommendations, implemented on the calculation module 3 described above.

The method is schematically represented on FIG. 4 and comprises the steps described below, in any technically feasible order. The method is implemented on the calculation module 3 described above and is described in view of the features of this calculator 3.

The method comprises an optional preliminary calibration step CAL, comprising the collection of values of the at least one positional parameter by the calculation module 3 and the sensors 4, and the selection of at least a part of the collected values to build a database of reference values of the positional parameter associated with a correct posture and/or a correct use of the optical element 7.

The method comprises a step OPT obtaining at least one value of at least one optical parameter representative of the optical element 7 used by the user. The value of the optical parameter is for example obtained from a database stored in the memory.

The value of the optical parameter may also be obtained based on measurements from at least one of the sensors 4, for example for determining a value of a variable parameter of the optical element 7. The value of the optical parameter used in the method is then selected form multiple values stored in the database based on associated measurements of the at least one sensor. The method comprises a step POS of obtaining at least one value of at least one positional parameter representative of a position, a movement or a succession of movements of at least one body-part of the user. This step is implemented on the calculation module 3 through the at least one sensor 4 as explained above.

The step of obtaining the values of the at least one positional parameter is, for example, implemented continuously or periodically.

The method may comprise an optional step SUP of obtaining at least one supplemental parameter, implemented on the calculation module 3 based on measurements from at least one of the sensors 4. The supplementary parameter may be related to the immediate environment of the user, such as a viewing distance, or be a contextual parameter, such as a location of the user or an activity being performed by the user.

The method comprises a step REF of selecting reference values for the at least one positional parameter based on the value of the at least one optical parameter. The reference values of the positional parameters may also be selected based on one or more supplemental parameters.

The method comprises a step COMP of comparing the values of the at least one positional parameter to the selected reference values to detect an inadequacy. The comparison step may also involve one or more supplementary parameters.

In embodiments, the method may also comprise a step of determining the selected reference values. This determination may be realised by selecting predetermined reference values, for example predetermined reference values of model users. The selection may be realised by comparing values of physiological parameters of the user with values of physiological parameters of the model users. The reference values may also be adapted according to evolution of the values of the physiological parameters of the user.

The method comprises a step REC of issuing at least one recommendation to the user in case of a detected inadequacy. The issuance of the inadequacy may be immediate, differed after the inadequacy has been maintained for a predetermined period, or be included in a periodic report including recommendations to the user.

The recommendation may be issued by any of the previously described means or any combination thereof.

In embodiments, the method may comprise a step of obtaining feedbacks of the user and adapting the method according to these feedbacks. The method may comprise one of the following actions according to these feedbacks:
continue running the method without modification
adapt the parameters of the method, for example, the optical parameters, and/or the positional parameters, or adapt the references values and continue running the method with the adapted parameters or the adapted reference values,
stop running the method.

1. A calculation module for evaluating visual and postural ergonomics of a user and issuing recommendations, the calculation module comprising:
at least one electronic circuit configured to:
obtain at least one value of at least one optical parameter representative of at least one optical element worn by the user;
obtain at least one value of at least one positional parameter representative of a position, a movement or a succession of movements of at least one body-part of the user
select reference values for the at least one positional parameter based on the value of the at least one optical parameter;
compare the values of the at least one positional parameter to the selected reference values to detect an inadequacy; and
issue at least one recommendation to the user in case of a detected inadequacy.

2. The calculation module according to claim 1, wherein the at least one positional parameter comprises at least one angle, direction or distance of observation of an area of interest by the user.

3. The calculation module according to claim 1, wherein the reference values comprise a range of adequate distances, angles, or directions of observation of an area of interest seen by the user through the optical element.

4. The calculation module according to claim 3, wherein the optical element is a progressive ophthalmic lens, the range of the adequate directions or the angles of observation of the area of interest being determined in relation to a distance to the area of interest and in relation to specific predetermined areas of the optical element.

5. The calculation module according to claim 3, wherein the area of interest comprises a display, the calculation module being configured to select the reference values also using a distance to the display.

6. The calculation module according to claim 5, wherein the display comprises at least one text area, the calculation module being configured to select the reference values also using a parameter representative of the text area.

7. The calculation module according to claim 1, wherein the at least one positional parameter comprises at least one head or neck position angle, the at least one reference parameter comprising a corresponding recommended angular range, the reference values of the at least one positional parameter further comprising a maximal duration spend out of the recommended angular range over a predetermined duration.

8. The calculation module according to claim 1, wherein the at least one positional parameter comprises a frequency of postural alteration, the postural alteration comprising a variation of at least one predetermined body-part positional angle by a predetermined minimum value, the reference values of the at least one the positional parameter comprising a minimum frequency of postural alteration, and/or
wherein the at least one positional parameter comprises a frequency of movement alteration, the movement alteration comprising a variation of at least one predetermined body-part movement by a predetermined minimum value, the reference values of the at least one positional parameter comprising a minimum frequency of movement alteration.

9. The calculation module according to claim 1, wherein the recommendation comprises a recommended displacement of an area of interest and/or a recommended displacement of the user relative to the area of interest.

10. The calculation module according to claim 1, wherein the recommendation comprises a postural alteration, the postural alteration comprising a variation of at least one predetermined body-part positional angle by a predetermined minimum value, a recommended break from a current activity and/or a modification of the optical element.

11. The calculation module according to claim 1, wherein the calculation module is configured to control at least one signaling device for issuing the recommendation, the signaling device comprising a visual device, an audio device, and/or an haptic device.

12. The calculation module according to claim 1, wherein the calculation module comprises a communication device for communicating with a computer, a smartwatch or a smartphone, for storing measurement data on the computer, the smartwatch or the smartphone and issuing notification through a program running on the computer, the smartwatch or the smartphone.

13. The calculation module according to claim 1, wherein the calculation module is also configured to use at least one environmental parameter comprising a nature of an activity performed by the user, the calculation module being configured to determine the activity being performed by the user based on measurements from at least one sensor, advantageously through an artificial intelligence-type algorithm implemented on the calculation module.

14. The calculation module according to claim 13, wherein the calculation module is configured to generate reference values of the at least one postural parameter and corresponding values of the at least one environmental parameter in a calibration step (CAL), the calibration step being triggered by the user or automatically.

15. A head mounted device comprising:
calculation circuitry; and
at least one optical element,
wherein the calculation circuitry is configured to:
evaluate visual and postural ergonomics of a user using the optical element and issue recommendations,
obtain, at least one value of at least one optical parameter representative of the optical element,
obtain, at least one value of at least one positional parameter representative of a position, a movement or a succession of movements of at least one body-part of the user,
select reference values for the at least one positional parameter based on the value of the at least one optical parameter,
compare the values of the at least one positional parameter to the selected reference values to detect an inadequacy, and
issue at least one recommendation to the user in case of a detected inadequacy.

16. A computer-implemented method for evaluating visual and postural ergonomics of a user and issuing recommendations, the method comprising
obtaining at least one value of at least one optical parameter representative of an optical element used by the user;
obtaining at least one value of at least one positional parameter representative of a position, a movement or a succession of movements of at least one body-part of the user;
selecting reference values for the at least one positional parameter based on the value of the at least one optical parameter;
comparing the values of the at least one positional parameter to the selected reference values to detect an inadequacy; and
issuing at least one recommendation to the user in case of a detected inadequacy.

17. The calculation module according to claim 2, wherein the reference values comprise a range of adequate distances, angles, or directions of observation of an area of interest seen by the user through the optical element.

18. The calculation module according to claim 3, wherein the area of interest comprises a display, the calculation module being configured to select the reference values also using a distance to the display.

19. The calculation module according to claim 2, wherein the at least one positional parameter comprises at least one head or neck position angle, the at least one reference parameter comprising a corresponding recommended angular range, the reference values of the at least one positional parameter further comprising a maximal duration spend out of the recommended angular range over a predetermined duration.

20. The calculation module according to claim 3, wherein the at least one positional parameter comprises at least one head or neck position angle, the at least one reference parameter comprising a corresponding recommended angular range, the reference values of the at least one positional parameter further comprising a maximal duration spend out of the recommended angular range over a predetermined duration.
